(19) United States
(12) Patent Application Publication
(10) Pub. No.: US 2025/0340505 A1
(43) Pub. Date: Nov. 6, 2025

(54) SOLID-STATE FORM OF HYDRATE OF CALCIUM OXYBATE, METHOD OF MAKING AND USE

(71) Applicant: Macfarian Smith Limited, Edinburgh (GB)

(72) Inventor: Ewart Grant, Devens, MA (US)

(21) Appl. No.: 18/253,588

(22) PCT Filed: Nov. 19, 2021
(86) PCT No.: PCT/IB2021/000800

§ 371 Date: May 18, 2023

Related U.S. Application Data
(60) Provisional application No. 63/198,888, filed on Nov. 20, 2020.

(51) Int. Cl. C07C 53/124; C07C 51/41; C07C 51/47

(52) U.S. Cl. CPC C07C 53/124; C07C 51/412; C07C 51/47

(57) ABSTRACT

The present invention is directed to Form A of monohydrate of calcium oxybate, a pharmaceutically acceptable composition thereof, its method of making, and use thereof.

### FIELD OF THE INVENTION

The invention relates to a hydrate of calcium oxybate, its method of making, and use thereof.

### BACKGROUND OF THE INVENTION

Oxybate, has the chemical designation, 4-hydroxybutanoate, having the following structure

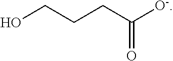

Sodium oxybate alone (XYREM®) is prescribed for the treatment of cataplexy or excessive daytime sleepiness (EDS) in patients 7 years of age and older with narcolepsy. A mixture of oxybate salts consisting of sodium, potassium, magnesium and calcium oxybate (XYWAV™) was approved for treating the same indications.

British Patent 922029 discloses 4-hydroxybutyric acid and it salts as having a sedative, hypnotic or anaesthetic effect in a patient and as being capable of putting patients into a sleep closely resembling ordinary sleep. The calcium salt of 4-hydroxybutyric acid is prepared by treating an aqueous solution of 4-butyrolactone (1 mole) with lime CaO (0.5 mole), followed by filtration, and then evaporating the clear filtered solution until the compound crystallizes. No XRPD, TGA, or DSC analyses are provided regarding the calcium oxybate prepared therein.

U.S. Pat. No. 4,393,236 discloses calcium oxybate as being a sleeping agent and a tranquilizer. The calcium oxybate is disclosed as being prepared by reacting a member of the group consisting of 4-hydroxybutyric acid, 4-butyrolactone and mixtures thereof with a member of the group consisting of calcium hydroxide, calcium oxide, calcium carbonate and mixtures thereof in an aqueous solution, and crystallizing the resulting solution at a temperature sufficiently low to produce a solid nonhygroscopic calcium oxybate salt, wherein before and/or during the crystallizing step a nonsolvent for the calcium 4-hydroxybutyrate can be added to the aqueous solution. Furthermore, the calcium oxybate salt can be recrystallized from an organic solution to provide nonhygroscopic, nonhydrated calcium-4-hydroxybutyrate. No XRPD, TGA, or DSC analyses are provided regarding the calcium oxybate prepared therein.

Forensic Science International 216 (2012) 158-162 discloses a dihydrate calcium oxybate salt. The dihydrate calcium oxybate salt is disclosed as being prepared by reacting calcium hydroxide suspended in water (25 mL) with gamma-butyrolactone, followed by removal of water to yield a viscous residue that is then mixed with acetone to yield the product following drying under heat. The primary peaks for the dihydrate calcium oxybate salt form are at 9.3, 18.6 and 23.9° 2θ.

### SUMMARY OF THE DISCLOSURE

The present invention is directed to Form A of monohydrate of calcium oxybate, a pharmaceutically acceptable composition thereof, its method of making, and use thereof.

### BRIEF DESCRIPTION OF THE DRAWINGS

FIG. 1 provides a representative XRPD pattern of Form A of monohydrate of calcium oxybate.

FIG. 2 provides a representative DSC plot of Form A of monohydrate of calcium oxybate.

FIG. 3 provides a representative TGA plot of Form A of monohydrate of calcium oxybate.

### DETAILED DESCRIPTION OF THE DISCLOSURE

The present invention is directed to Form A of monohydrate of calcium oxybate, a pharmaceutically acceptable composition thereof, its method of making, and use thereof.

As used herein and unless otherwise specified, the term “solid-state form” includes crystalline or polymorphic forms, amorphous phase, and solvates.

As used herein and unless otherwise specified, the terms “about” and “approximately,” when used in connection with a numeric value or a range of values which is provided to characterize a particular solid form, e.g., a specific temperature or temperature range, such as, e.g., that describing a DSC or TGA thermal event, including, e.g., melting, dehydration, desolvation or glass transition events; a mass change, such as, e.g., a mass change as a function of temperature or humidity; a solvent or water content, in terms of, e.g., mass or a percentage; or a peak position, such as, e.g., in analysis by IR or Raman spectroscopy or XRPD; indicate that the value or range of values may deviate to an extent deemed reasonable to one of ordinary skill in the art while still describing the particular solid form.

As used herein and unless otherwise specified, the term “crystalline” and related terms used herein, when used to describe a compound, substance, modification, material, component or product, unless otherwise specified, mean that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, Md. (2005); The United States Pharmacopeia, 23rd ed., 1843-1844 (1995).

As used herein and unless otherwise specified, the terms “polymorph,” “polymorphic form” or related term herein, refer to a crystal form of an API (active pharmaceutical ingredient) free base or salt thereof that can exist in two or more forms, as a result of different arrangements or conformations of the molecule, ions of the salt, or addition and arrangement of solvents within the crystalline lattice.

As used herein and unless otherwise specified, the terms “substantially” or “substantially free/pure” with respect to a polymorph or polymorphic form means that the form contains about less than 30 percent, about less than 20 percent, about less than 15 percent, about less than 10 percent, about less than 5 percent, or about less than 1 percent by weight of impurities. Impurities may, for example, include other polymorphic forms, water and solvents other than that in a solvated crystalline polymorphic form.

As used herein and unless otherwise specified, the abbreviations: “EtOH” refers to ethanol; “i-PrOH” refers to isopropanol; “TBME” refers to tert-butyl methyl ether; and “GBL” refers to gamma-butyrolactone.

As used herein and unless otherwise specified, the term “pre-washed celite” refers to diatomaceous earth washed with water, and optionally, ethanol, filtered, and optionally, dried. In a particular embodiment, the celite is dried after washing to reduce yield loss due to excess water in the celite.

Techniques for characterizing crystal and amorphous forms include but are not limited to differential scanning calorimetry (DSC), thermal gravimetric analysis (TGA), X-ray powder diffractometry (XRPD), and Karl Fischer (KF).

Thermogravimetric analysis (TGA) data is collected using a TA Instruments Q500 equipped with a single position sampler. Samples of about 2-10 mg are placed into a sealed, pre-tared, aluminum pan with a pin hole in the lid to allow gases to escape. Samples are scanned from about 25° C. to about 300° C. at the rate of about 10° C./min using a nitrogen purge at about 60 mL/min.

X-ray powder diffraction (XRPD) patterns are obtained using a Bruker D8 Advance equipped with a Cu Kα radiation (40 kV, 40 mA) source (λ=1.54° A), a 9-position sample holder, and a θ-2θ goniometer fitted with a Ge monochromator. The incident beam is passed through a 2.0 mm divergence slit followed by a 0.2 mm anti-scatter slit. The diffracted beam is passed through an 8.0 mm receiving slit with 2.5° Soller slits followed by a LYNXEYE super speed detector. Samples are prepared on polished, zero-background silicon plates and run using a low background, airtight specimen dome. A 30-minute method and the angular range of 3 to 40° with a step size of 0.018° 2θ at 0.85 seconds per step is applied in using the Bruker D8 Advance. Diffrac Plus XRD Commander and Diffrac Plus EVA software, respectively are used for data collection and analysis. One skilled in the art would recognize that the °2θ values and the relative intensity values are generated by performing a peak search on the measured data and that the d-spacing values can be calculated by the instrument from the °2θ values using Bragg's equation. One skilled in the art would further recognize that the relative intensity for the measured peaks may vary as a result of sample preparation, orientation and instrument used, for example.

Differential scanning calorimetry (DSC) samples are collected using a TA Instruments Q2000 equipped with an auto-sampler and RCS40 refrigerated cooling system. Samples of 2-5 mg are hermetically sealed in Tzero aluminum pans with matching lids and scanned from 25° C. to 350° C. at a ramp rate of 10° C./min in T4P mode under a nitrogen purge of 50 mL/min.

A Mettler Toledo DL39 Coulometric Karl Fischer (KF) Titrator is used to determine the apparent water content in samples. About 10 mg of the solid is used for titration. To limit the exposure of the solution to the air, the titrator opening is closed by a stopper immediately after adding samples. HYDRANAL-Coulomat AD is used as the titrant added at 40% speed. A mixing time of 30 seconds is used prior to analysis.

KF data is measured using a Mettler Toledo DL32 Karl Fisher (KF) coulometer. Solid samples (about 5-10 mg) are weighted into a weighing funnel that is used to transfer the material into a titration vessel. To limit the exposure of the solution to the air, the titrator opening is closed by a stopper immediately after adding samples. The sample is mixed for 30 seconds prior to analysis.

In a method according to the invention, Form A of monohydrate of calcium oxybate is prepared comprising:
a) reacting an aqueous slurry of calcium hydroxide with GBL at an elevated temperature to form an aqueous solution of calcium oxybate;
b) cooling the reaction mixture of step a);
c) adding EtOH to the aqueous solution of calcium oxybate;
d) adding celite to the EtOH and aqueous solution of calcium oxybate of step c);
e) filtering the EtOH and aqueous solution of calcium oxybate of step d) containing celite to collect a calcium oxybate containing filtrate; and
f) adding i-PrOH to the calcium oxybate containing filtrate to yield the Form A of monohydrate of calcium oxybate.
   In one embodiment, the molar equivalent ratio of GBL to calcium hydroxide is about 1.85-2.05:1, particularly about 1.95:1. In another embodiment, about 1.42-2.0 volumes, particularly about 1.5 volumes (mL), of water are used per weight (g) of GBL. In one embodiment, the elevated temperature is about 65-85° C., particularly at about 70-80° C. In one embodiment, the aqueous slurry of calcium hydroxide is heated to the elevated temperature before reacting with GBL. In yet another embodiment, the reacting is effected by combining the GBL with the aqueous slurry of calcium hydroxide over about 3-5 h; particularly over about 4 h. In another embodiment, the reacting further comprises holding the mixture for about 4-24 h. In a particular embodiment, the mixture is held at the elevated temperature. In another embodiment, the cooling is effected at about 20-30° C., particularly at about 20-25° C. In another embodiment, about 1.8-2.2 volumes, particularly about 2 volumes (mL), of EtOH are added to the aqueous solution of calcium oxybate per weight (g) of GBL. In a particular embodiment, the ethanol is SDA 2B-3 ethanol which contains about 0.5% toluene. In another embodiment, the ratio of celite added is about 0.2 g to 1 g of GBL. In one embodiment, the reaction mixture is stirred for about 15-20 minutes after addition of the celite. In a particular embodiment, the celite of step d) is pre-washed celite. In one embodiment, the filtering is through a filter containing celite, which in a particular embodiment is pre-washed. In another embodiment, the filter is washed with about 0.9-1.1 volumes, particularly about 1 volume (mL), of ethanol per weight (g) of GBL. In another embodiment, the filtrate is additionally filtered using a 0.45 to 1-micron filter. In another embodiment, about 6.3-9 volumes, particularly about 7 volumes (mL), of i-PrOH per weight (g) of GBL is added to the filtrate. In one embodiment, the i-PrOH is added over about 3-5 hours, particularly over about 4 hrs. In another embodiment, the i-ProH is added in stages with a first portion being added to a cloud point, followed by a hold period, followed by addition of a second portion. In one embodiment, the i-PrOH is added to the filtrate at about 15-25° C., particularly at about 20-22° C. In one embodiment, the mixture of i-PrOH and filtrate is stirred at about 15-25° C., particularly at about 20-22° C., for at least about 1 hr to yield Form A of monohydrate of calcium oxybate. In another embodiment, the formed Form A of monohydrate of calcium oxybate is isolated and washed at least once, preferably twice, with i-PrOH. In one embodiment, about 2 volumes of i-PrOH (mL) are used for the wash per weight (g) of GBL. In one embodiment, the formed Form A of monohydrate of calcium oxybate is washed at least once, preferably twice, with about 2 volumes of TBME (mL) per weight (g) of GBL. In a particular embodiment, the formed Form A of monohydrate of calcium oxybate is not washed with TBME. In another embodiment, the isolated Form A of monohydrate of calcium oxybate is dried at about 50° C. under reduced pressure, for example, at about 200 Torr.

### EXAMPLES

Examples 1 and 2, which follow herein, provide embodiments of the preparation of Form A of monohydrate of calcium oxybate.

The examples are presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles described herein may be applied to other applications without departing from the spirit and scope of the various embodiments. Therefore, the various embodiments are illustrative of the present disclosure and the disclosure is not intended to be limited to the examples described herein and shown.

### Example 1

### Preparation of Form A of Monohydrate of Calcium Oxybate

Water (1.5 g) is charged to a reaction vessel. 0.43 g (1.0 equiv., 5.8 mmol) of calcium hydroxide is charged to the reaction vessel. The mixture is heated to 75±2° C., and 1 g (1.95 equiv., 11.6 mmol) gamma-butyrolactone (GBL) is charged to the reaction mixture over 4 hr at 75±5° C. The mixture is then held at that temperature for 4-24 h. The reaction is tested for completion using UPLC as described below.

When reaction is complete, the mixture is cooled to 20-25° C. 1.6 g of SDA 2B-3 (ethanol with 0.5% toluene) (2 volumes (mL) per g of GBL) is added to the cooled mixture, and then 0.2 g of prewashed celite is added thereto. The mixture with the celite is stirred for 15-20 min. The mixture is filtered through a filter containing prewashed celite to collect the filtrate. 0.80 g of SDA 2B-3 (1 volume (mL) per g of GBL) is used to rinse the reaction vessel and then to wash the filter, and is added to the filtrate. The filtrate is further filtered through a 0.45 to 1-micron filter. To the resulting filtrate is added 5.5 g isopropanol (7 volumes (mL) per g of GBL) over 4 hr at 20-22° C. The resultant mixture is stirred at 20-22° C. for minimum of 1 hr to precipitate a solid. The solid is isolated by filtration, washed two times with 1.6 g i-PrOH (2 volumes (mL) per g of GBL), and then washed two times with 1.5 g TBME (2 volumes (mL) per g of GBL). The solid is then dried at 50° C./200 Torr to yield Form A of monohydrate of calcium oxybate.

XRPD 20 pattern peaks and relative % intensity values for the peaks of Form A of monohydrate of calcium oxybate are shown in Table I.

TABLE 1
Average Peak List for Form A of monohydrate of calcium oxybate

| Angle (2Θ°) | Intensity |
|---|---|
| degree | % |
| 7.2 | 100 |
| 8.1 | 8.1 |
| 13.7 | 0.8 |
| 14.2 | 6 |
| 14.7 | 1.3 |
| 16.2 | 1.1 |
| 22.7 | 1.2 |
| 24.3 | 0.8 |
| 26.0 | 1.7 |
| 28.5 | 1.1 |
| 29.0 | 0.9 |
| 32.7 | 2.4 |
| 36.0 | 2.5 |
| 39.7 | 1.4 |

The angle measurements are ±0.2° 2θ. Key defining peaks for solid-state Form A of monohydrate of calcium oxybate include one or two of 7.2 and 8.1° 2θ, and optionally in addition thereto 14.2° 2θ.

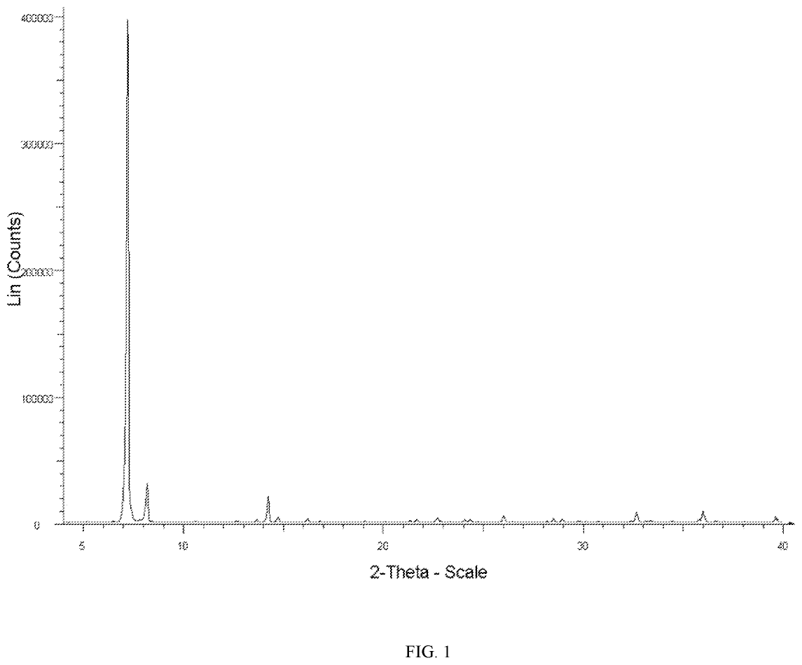

FIG. 1 is a representative XRPD pattern of Form A of monohydrate of calcium oxybate.

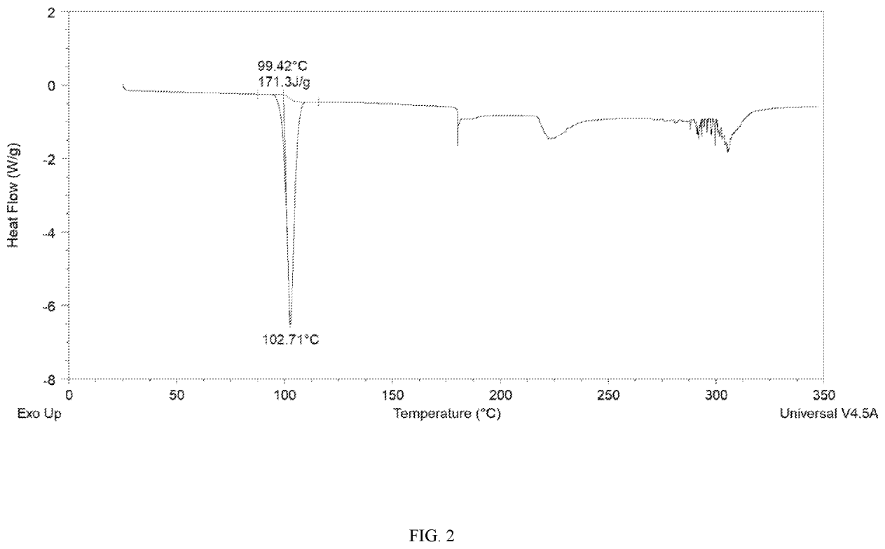

FIG. 2 is a representative DSC of Form A of monohydrate of calcium oxybate which shows an endotherm with an onset of around 99° C. that is associated with partial dehydration.

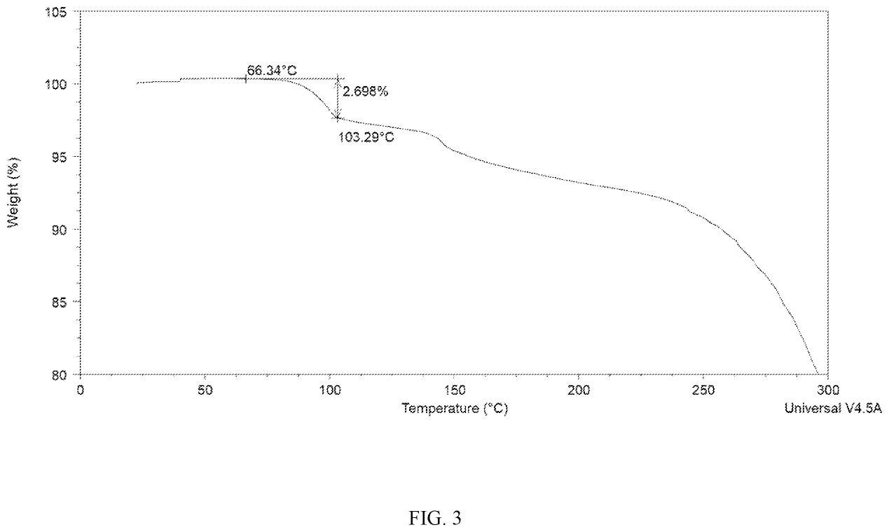

FIG. 3 is a representative TGA of Form A of monohydrate of calcium oxybate which shows a partial loss of hydrate starting at about 66° C. with a total weight loss of about 2.7% by about 103° C., followed by slow decomposition and loss of the remaining water.

The water content is determined by averaging KF data on 3 samples and is shown to be 6.7%±1.0% for Form A of monohydrate of calcium oxybate.

### UPLC Reaction Completion Test

Reaction is complete when the level of GBL is <0.5 area % and the level of Oxybate Impurity 1 is <0.2 area %. Oxybate Impurity 1 has the following structure and a relative retention time (RRT) of about 3.65-3.70:

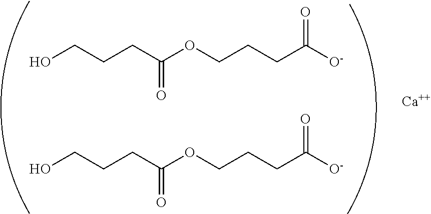

The RRT of Ca oxybate is 1.0 and the RRT of GBL is about 1.35.
UPLC: System equipped with TUV detection, column temperature control, electronic data collection and processing, or equivalent
Column: Waters Acquity HSS T3, 1.8 μm, 150×2.1 mm
Balance: Analytical balance capable of weighing to ±0.1 mg

### Class A Volumetric Flasks and Pipettes

Diluent 1: Water; Diluent 2: 1% MSA in Water (5.0 mL of methane sulfonic acid in 500 mL water and mixed well).

### Mobile Phase:

Mobile Phase A: Sodium Phosphate Monobasic Buffer (1.0 g of sodium phosphate monobasic in 1000 mL water, 2.0 mL of phosphoric acid is added and mixed well).
Mobile Phase B: 95:5 Acetonitrile: Water (50 mL of water in 950 mL of acetonitrile and mixed well).

### Sample

Prepare the sample solution at approximately 25 mg/mL.

Accurately weigh approximately 125 mg of sample and quantitatively transfer to a 5-mL volumetric flask. Add 2.5 mL of Diluent 2 (1% MSA in water) and sonicate to dissolve, if necessary. Dilute to volume with Diluent 2 and mix well. Prepare in duplicate.

Chromatographic Conditions

| Column: | Acquity HSS T3, 1.8 μm, 150 × 2.1 mm | |
|---|---|---|
| Flow Rate: | 0.2 | mL/min |
| Detection Wavelength: | 215 | nm |
| Column Temperature: | 40° | C. |
| Sample Temperature: | 5° | C. |
| Injection Volume: | 2.5 | μL |
| Mobile Phase A: | Sodium Phosphate Monobasic Buffer | |
| Mobile Phase B: | 95:5 Acetonitrile:Water | |
| Data Collection: | 25.0 | minutes |
| Equilibration Time: | 10.0 | minutes |
| Run Time: | 35.0 | minutes |

### Example 2

### Preparation of Form A of Monohydrate of Calcium Oxybate

Calcium hydroxide (385 g, 98.8% assay, 0.51 equiv.) and deionized water (1724 mL, 2.0 vol.) are charged to a 5-liter glass reactor with overhead stirring and reflux condenser and stirred to form a slurry. The batch is heated to 70-80° C. GBL (861 g, 1.0 equiv.) is added to the batch slowly over 3 hours, 5 minutes. The batch is held at 70-80° C. for approximately 17 h. The reaction is tested for completion using UPLC as described herein.

The batch is cooled to 20-30° C. Celite 545 (172 g) and an SDA 2B-3 alcohol (1727 mL, 2.0 vol.) are charged to the batch. Celite 545 (56 g) is slurried in 300 mL deionized water and loaded on to a 25-50-micron filter frit. The water is drained with vacuum to form a Celite bed. The batch is filtered through the Celite 545 bed on a 25-50-micron frit. This filtration is very slow and after ⅔ of the batch is filtered it has to be held incomplete overnight. The Celite bed is hard and impenetrable the next morning. The Celite bed is slurried in 10 mL DI water to loosen it up and returned to the filter. The remainder (⅓) of the batch is heated to 30° C. and passed through the Celite bed and combined with the previous day's filtrate. The batch is passed through Sharkskin (8-12-micron cellulose) filter media and it is still hazy. The batch is passed through a double layer of Sharkskin filter media and it is still hazy. The batch is passed through two-ply 5-micron polyester woven cloth and it is still hazy. The batch is passed through 1.5-micron glass fiber filter (Whatman 934-AH) and it is still hazy. The batch is passed through a 1-micron absolute filter membrane and following this filtration it is clear. The batch is transferred to a 20-liter straight side reactor with overhead stirring. The reactor & filter are rinsed with SDA 2B-3 alcohol (869 mL, 1.0 vol.) to complete the transfer. Isopropanol (7754 mL, 9.0 vol.) is added to the batch over about 3 hours. The product precipitates during this addition. The batch slurry is agitated for about 14 hours at 15-25° C. The product is collected on 5-micron polyester cloth. The filter cake is washed twice with isopropanol (1722 mL each, 2.0 vol.) and then twice with tert-butyl methyl ether (1724 mL each, 2.0 vol.). The filter cake is pulled down with vacuum for about 3 hours. The solid is loaded into a dryer and dried at 50° C. & approx. 10 mbar vacuum for 5 days.

The above examples are set forth to aid in the understanding of the disclosure and are not intended and should not be construed to limit in any way the disclosure set forth in the claims which follow hereafter.

1. A compound which is Form A of monohydrate of calcium oxybate.

2. The compound of claim 1 which is characterized by having one or more X-ray powder diffraction peaks selected from about 7.2 and 8.1°, and 14.2 2θ±0.2° 2θ as measured by CuKα radiation.

3. (canceled)

4. The compound of claim 1 which is characterized by onset of an endothermic event at about 99° C.±3° C., as measured by differential scanning calorimetry.

5. A pharmaceutical composition comprising the compound of claim 1 and a pharmaceutically acceptable excipient.

6. (canceled)

7. A method for preparing the compound of claim 1 comprising:
a) reacting an aqueous slurry of calcium hydroxide with GBL at an elevated temperature to form an aqueous solution of calcium oxybate;
b) cooling the reaction mixture of step a);
c) adding EtOH to the aqueous solution of calcium oxybate;
d) adding celite to the EtOH and aqueous solution of calcium oxybate of step c);
e) filtering the EtOH and aqueous solution of calcium oxybate of step d) containing celite to collect a calcium oxybate containing filtrate; and
f) adding i-PrOH to the calcium oxybate containing filtrate to yield the Form A of monohydrate of calcium oxybate.

8. The method of claim 7 wherein the molar equivalent ratio of GBL to calcium hydroxide is about 1.85-2.05:1.

9. The method of claim 7 wherein about 1.42-2.0 volumes (mL) of water are used per weight (g) of GBL.

10. The method of claim 7 wherein the elevated temperature is about 65-85° C.

11. (canceled)

12. (canceled)

13. (canceled)

14. The method of claim 7 wherein the cooling in step b) is affected at about 20-30° C.

15. The method of claim 7 wherein about 1.8-2.2 volumes (mL) of EtOH are added to the aqueous solution of calcium oxybate per weight (g) of GBL.

16. The method of claim 7 wherein the EtOH contains about 0.5% toluene.

17. The method of claim 7 wherein the ratio of celite added is about 0.2 g to 1 g of GBL.

18. (canceled)

19. The method of claim 7 wherein the filtering of step e) is through a celite filter.

20. (canceled)

21. The method of claim 7 further comprising washing the filter with about 0.9-1.1 volumes (mL) EtOH per weight (g) of GBL before conducting step f).

22. The method of claim 7 further comprising additionally filtering the filtrate of step e) before conducting step f) using a 0.45 to 1-micron filter.

23. The method of claim 7 wherein about 6.3-9 volumes (mL) of i-PrOH per weight (g) of GBL is added to the filtrate.

24. The method of claim 7 wherein the i-PrOH is added over about 3-5 hours.

25. The method of claim 7 wherein the i-PrOH is added to the calcium oxybate containing filtrate at about 15-25° C.

26. The method of claim 7 further comprising stirring the mixture of i-PrOH and calcium oxybate containing filtrate at about 15-25° C. for at least about 1 hr to yield the Form A of monohydrate of calcium oxybate.

27. (canceled)

28. The method of claim 27 further comprising washing the isolated Form A of monohydrate of calcium oxybate with about 2 volumes of i-PrOH (mL) per weight (g) of GBL.

29. The method of claim 27 further comprising drying the isolated Form A of monhydrate of calcium oxybate at about 50° C. under reduced pressure.
